(19) United States
(12) Patent Application Publication
(10) Pub. No.: US 2025/0127745 A1
(43) Pub. Date: Apr. 24, 2025

(54) NOVEL COMPOSITIONS FOR PROMOTING EYE HEALTH OF A COMPANION ANIMAL

(71) Applicant: Bausch + Lomb Ireland Limited, Dublin (IE)

(72) Inventors: Albert Jernigan Swann, Knoxville, TX (US); Carina Norrene Gassman, Rochester, NY (US)

(21) Appl. No.: 18/923,869

(22) Filed: Oct. 23, 2024

Related U.S. Application Data
(60) Provisional application No. 63/545,288, filed on Oct. 23, 2023.

(51) Int. Cl. A61K 31/202; A61K 9/00; A61K 31/015; A61K 31/047; A61K 31/201; A61K 35/60; A61K 36/185; A61K 36/23; A61K 36/28; A61K 36/286; A61K 36/64

(52) U.S. Cl. CPC A61K 31/202; A61K 9/0056; A61K 31/015; A61K 31/047; A61K 31/201; A61K 35/60; A61K 36/23; A61K 36/28; A61K 36/286; A61K 36/577; A61K 36/64

(57) ABSTRACT

The present invention relates to a shelf-stable veterinary formulation for promoting eye health and/or comfort in a companion animal consisting of a palatable ductile chewable veterinary composition comprising an omega-3 fatty acid oil; at least one anti-inflammatory plant extract; and at least one carotenoid. The present invention also relates to methods of promoting eye health and/or comfort in a companion animal using such compositions.

### CROSS REFERENCE TO RELATED APPLICATIONS

This application claims the benefit of provisional U.S. Application No. 63/545,288 filed Oct. 23, 2023, the disclosure of which is incorporated by reference herein in its entirety.

### FIELD OF THE INVENTION

The present invention relates to a shelf-stable veterinary formulation for promoting eye health and/or comfort in a companion animal consisting of a palatable ductile chewable veterinary composition comprising an omega-3 fatty acid oil; at least one anti-inflammatory plant extract; and at least one carotenoid. The present invention also relates to methods of promoting eye health and/or comfort in a companion animal using such compositions.

### BACKGROUND OF THE INVENTION

As companion animals such as dogs age, they may experience changes in their vision and eye health. In addition, certain breeds of dogs may be more prone to eye problems such as dry and irritated eyes. In addition to cataracts and other ocular conditions that can affect visual acuity, canines may often suffer from eye irritation and other discomfort including (1) dryness; (2) burning; (3) itching; (4) irritation; (5) redness; and/or (6) foreign body sensations. Such irritations can be the result of environmental factors such as exposure to smoke, wind, dust, or other environmental irritants; dietary factors such as a diet containing pro-inflammatory ingredients; or physiological factors such as a poor quality or quantity of the canine's lipid layer in the eye. Regardless of the underlying cause, eye irritation not only causes stress to the animal, but also to the human caretaker of that animal.

Nutritional supplements can play an important role in promoting and maintaining eye health and/or comfort in dogs. However, optimal levels of ingredients that may be useful in nutritional supplements promoting the eye health of a companion animal have not been combined in a convenient, shelf-stable, and palatable form that is readily and eagerly consumed with minimal intervention of a human caretaker.

Previous supplements for humans and or companion animals have come in the form of high protein “treats,” soft chews, capsules, tablets, gel caps, liquids, kibble, gravy, or other dry or wet feed additives; all of which may present one or more drawbacks in stability, nutritional availability, and/or administration. Administering supplements in, in for example, gel cap, capsule, or dry tablet form may pose a choking hazard, particularly for smaller dogs. This creates a need for a nutritional supplement that is not only remains palatable in a shelf-stable form, but also easy to administer to dogs of all sizes.

Some formulations may have a taste or odor that is unpleasant to canines, leading to low palatability and resistance from the dogs to take them. Even formulations that may be palatable to a vast majority of dogs, might be rejected by a “picky” dog. This can make it challenging and frustrating for those pet owners to ensure their pets receive the necessary supplements to maintain their eye health. In fact, the existence of “picky” individuals is surprising in a species not known for its culinary discernment (anecdotal reports indicate that many companion dogs will eagerly consume any non-canine excrement they encounter). But this very contraction of a picky individual in a non-selective species can cause significant frustration in pet owners when a supplement that may be accepted by greater than 95% of dogs, is entirely rejected by their dog. More preferable would be a shelf-stable supplement that is at least partially accepted by even the pickiest of dogs, despite those same dogs occasionally rejecting such supplements.

The ability of a product to maintain its palatability, safety, and efficacy over time when stored under normal conditions (often referred to as shelf stability) is important for nutritional supplements because these products are typically consumed over a period of weeks, months, or even years, and they must retain their nutritional value and palatability during this time.

If a nutritional supplement is not shelf-stable, it may degrade or lose its effectiveness over time due to factors such as exposure to light, heat, moisture, or oxygen. This can lead to reduced efficacy, poor product performance, or even safety concerns. In some cases, a supplement that has lost its potency may even be harmful to consume.

Degradation of the components of a nutritional supplement may also impact its palatability. Palatability, in this context, refers to how appealing or enjoyable a food or nutritional supplement is to the senses of a companion animal, including taste, smell, texture, and appearance. When a product loses its quality and taste due to poor shelf stability, it can become less palatable and less appealing to the companion animal.

For example, the degradation of ascorbic acid, also known as vitamin C, can affect the taste of food and beverages that contain it. Ascorbic acid is an antioxidant that is commonly added to foods and beverages as a preservative to prevent oxidation and improve shelf life. However, when ascorbic acid degrades, it has the potential to produce off-flavors and odors that can affect the taste and smell of the product to a companion animal.

When oxygen is present in the system, ascorbic acid is degraded primarily to dehydro ascorbic acid (DHAA). DHAA is not stable and spontaneously converts to 2,3-diketo-I-gulonic acid. In the presence of moisture, ascorbic acid can be degraded via multiple pathways. Some pathways result in formation of L-threonate, which can have unpredictable impacts on palatability. In fact, some humans have described the taste of this chemical as “unpleasantly soapy” while others have praised its “donut-like” flavor. It is not known which words dogs would choose to describe the flavor of this compound, but the variability in human perception perhaps hints as a basis for why certain individual dogs may react to supplements more negatively than their canine peers. Moreover, threonate can be oxidized to form L-tartrate, which is almost universally perceived as an extreme tart flavor.

The degree to which the degradation of ascorbic acid affects the taste of a product can vary depending on the concentration of the acid, the specific degradation products that are formed, and the other ingredients in the product. In some cases, the degradation of ascorbic acid may be barely noticeable, if at all, while in others it may result in a significant change in taste and aroma. This change in taste in aroma may be perceptible by only a small proportion of a population, causing significant frustration to those pet owners who cannot support their companion animal's ocular health.

In addition, water activity (the amount of free or unbound water and designated as a_{w}) in a edible product can affect the shelf life of nutritional supplements for companion animals. Water activity is a thermodynamic property of the food material that measures the ratio of free water in the food material vs. the total amount of water in the food material. If the amount of free water is the same as the total amount of water in the food material, the a_{w} is equal to 1. Water activity is measured by a commercially available water activity meter. Such meters are known to those skilled in the art, for example the PawKit meter. If all of the water in the material is bound to other components in the food material, the water activity is very low, and thus unavailable to microbes and enzymes with may affect the stability of a supplement. Most bacteria do not grow at water activities below 0.91, and most molds do not grow at water activities below 0.80. Therefore, by measuring water activity, it is possible to predict the shelflife of food products. For example, meat products have high palatability to companion dogs but also have a moisture content, thus their water activity lies in the upper range of the water activity scale for foods and may suffer from stability issues in the form of spoilage, palatability decline, and/or potency decline.

However, a composition with a very low a_{w} may be more stable, but may lack palatability to canines who do not enjoy the overly dry texture or it may suffer from decreased ease of administration because the texture may lack cohesion and become crumbly.

Therefore, there is a need for an eye health nutritional supplement for dogs that overcomes the disadvantages in the palatability of known compositions yet still providing eye health promoting levels of ingredients in an easy to administer form. Such a supplement should be highly palatable, easy to administer, shelf-stable, and safe for all dog sizes. By addressing these concerns, the supplement can help promote and maintain eye health in dogs in a more effective and convenient way, improving their quality of life and ensuring that they receive the necessary nutrients for their ocular well-being.

### DETAILED DESCRIPTION OF THE INVENTION

The present inventors have surprisingly discovered that improved, stable palatable compositions for promoting eye health and/or comfort in a companion animal comprising (1) at least one omega-3 fatty acid oil; (2) at least one anti-inflammatory plant extract; and (3) at least one carotenoid. The supplement is shelf-stable, efficacious in promoting the eye health of companion animals, and palatable to “picky” dogs.

These and other features, aspects, and advantages of the disclosure will be apparent from a reading of the following.

Definitions of certain terms as used in this application are provided below. Unless defined otherwise, all technical and scientific terms used herein have the normal and common meaning that would be commonly understood by one of ordinary skill in the art to which this disclosure belongs.

Moreover, as used herein, the term “about” means that dimensions, sizes, formulations, parameters, shapes and other quantities and characteristics are not and need not be exact, but may be approximate and/or larger or smaller, as desired, reflecting tolerances, conversion factors, rounding off, measurement error and the like, and other factors known to those of skill in the art. In general, a dimension, size, formulation, parameter, shape or other quantity or characteristic is “about” or “approximate” whether or not expressly stated to be such. It is noted that embodiments of very different sizes, shapes and dimensions may employ the described arrangements.

All embodiments described herein that encompass the invention can, in alternate embodiments, be more specifically defined by any of the transitional terms “comprising,” “consisting essentially of,” and “consisting of.”

As used herein, the term “essentially free of” means, on a weight basis, containing at most a trivial amount (e.g., less than 0.1 percent, less than 0.01 percent, less than 0.001 percent, or even less than 0.0001 percent) or completely free of.

Another embodiment of the present invention is a palatable composition for promoting the eye health of a companion animal comprising: (a) omega-3 fatty acid oil; (b) at least one anti-inflammatory plant extract; and (c) at least one carotenoid. The omega-3 fatty acid oil can be or contain one or more of eicosapentaenoic acid (EPA) and docosapentaenoic acid (DHA). In a preferred embodiment, the omega-3 fatty acid oil comprises at least two omega-3 fatty acids, including eicosapentaenoic acid (EPA) and a second omega-3 fatty acid is docosapentaenoic acid (DHA). The omega-3 fatty acid oil may also contain an omega-6 fatty acid, such as linoleic acid

The compositions of the invention comprise an omega-3 fatty acid oil comprising one or more omega-3 fatty acids.

As used herein, “omega-3 fatty acid” includes natural or synthetic, free omega-3 fatty acids and derivatives thereof, unless expressly excluded. Similarly, reference to a particular omega-3 fatty acid includes derivatives thereof, unless expressly excluded.

Examples of omega-3 fatty acids include:
a) omega-3 polyunsaturated free fatty acids (e.g., having aliphatic tails of 12-22 carbons, especially 18-22 carbons), including omega-3 polyunsaturated, long-chain free fatty acids, e.g., eicosapentaenoic acid C20:5 (“EPA”), docosahexaenoic acid C22:6 (“DHA”), and alpha-linolenic acid;
b) esters of omega-3 fatty acids (e.g. omega-3 polyunsaturated free fatty acids, e.g., long chain) with glycerol such as mono, di and triglycerides; and
c) esters of omega-3 fatty acids (e.g. omega-3 polyunsaturated free fatty acids, e.g. long chain) and a primary, secondary or tertiary alcohol such as fatty acid methyl esters and ethyl esters.

The omega-3 fatty acid oil may comprise a mixture any of the foregoing.

Preferred omega-3 fatty acids are polyunsaturated long chain free fatty acids, triglycerides thereof, ethyl esters thereof, and mixtures thereof, including for example EPA, DHA, triglycerides of EPA, triglycerides of DHA, ethyl esters of EPA, ethyl esters of DHA and mixtures thereof.

The omega-3 fatty acid oil may comprise an omega-3 fatty acid in its pure form or e.g., as a component of an oil such as marine or botanical oils (including concentrates thereof), such as fish oil (also known as marine oil), preferably purified fish oil concentrates, perilla oil, marine microalgae oil, or the oil from flax (linseed), chia, kiwifruit, lingonberry, camelina, purslane, or black raspberry seed. Oils containing high concentrations of omega 3 fatty acids such as fish oils or their mixtures, including concentrates thereof, are particularly useful. The term “omega-3 fatty acid oil” encompasses these forms.

In some embodiments, the omega-3 fatty acid oil comprises a mixture of fatty acids. In various embodiments, the fatty acids may be only omega-3 fatty acids or may comprise one or more omega-3 fatty acids in combination with one or more non-omega-3 fatty acids, e.g., omega-6 fatty acids, omega-9 fatty acids, etc. In some embodiments the mixed fatty acid composition comprises fatty acids in a concentration of at least 25% by weight, preferably at least 40% by weight, more preferably at least 50% by weight, still more preferably at least 60% by weight, still more preferably at least 70% by weight, most preferably at least 80% by weight, or even at least 90% by weight, by weight of the composition.

In some embodiments the omega-3 fatty acid oil comprises one or more omega-3 fatty acids in a total concentration of at least 25% by weight, preferably at least 40% by weight, more preferably at least 50% by weight, still more preferably at least 60% by weight, still more preferably at least 70% by weight, most preferably at least 80% by weight, or even at least 90% by weight (e.g. at least 95%, to 100% by weight), by weight of the total fatty acids in the oil. The composition may comprise pure omega-3 fatty acid(s).

Preferably, the omega-3 fatty acid oil comprises at least 50% by weight of EPA and DHA, more preferably at least 60% by weight, still more preferably at least 70% by weight, most preferably at least 80%, such as about 84% by weight, by weight of the total fatty acids in the oil.

The omega-3 fatty acid oil may comprise about 5 to about 100% by weight, more preferably about 25 to about 75% by weight, still more preferably about 40 to about 55% by weight, and most preferably about 46% by weight of EPA by weight of the total fatty acids in the oil.

The omega-3 fatty acid oil may comprise about 5 to about 100% by weight, more preferably about 25 to about 75% by weight, still more preferably about 30 to about 60% by weight, and most preferably about 38% by weight of DHA by weight of the total fatty acids in the oil.

In other embodiments, the percentages above are by weight of the omega-3 fatty acid oil.

The percentages by weight may be based on the free acid or ester forms, although it is preferably based on the ethyl ester form of the omega-3 fatty acids (and other fatty acids, if present) even if other forms are utilized in accordance with the present invention. As used herein, reference to “EPA” and “DHA” include the free acid and ester forms (including specifically, e.g., the ethyl ester forms), unless otherwise stated.

In some embodiments, the EPA and DHA are in a weight ratio of EPA: DHA of from 99:1 to 1:99, e.g., from 1:4 to 4:1, e.g. from 1:3 to 3:1, or e.g. from 1:2 to 2:1. The omega-3 fatty acid oil may also comprise pure EPA or pure DHA.

In some embodiments, the omega-3 fatty acid oil is a fish oil containing omega-3 acids as described in the US Pharmacopeia (“USP”) and National Formulary, Dietary Supplements, e.g. USP32-NF7, released Nov. 1, 2008, official date May 1, 2009, and/or as supplemented in the Second Supplement (released Jun. 1, 2009, official date Dec. 1, 2009); The United States Pharmacopeial Convention; United Book Press Baltimore MD.

In some embodiments, the omega-3 fatty acid oil is obtained from the body oil of fish families such as Engraulidae, Carangidae, Clupeidae, Osmeridae, Salmonidae, and Scombridae. In some embodiments the fish oil is purified, e.g., using urea fractionation followed by molecular distillation. In some embodiments, including where obtained from the foregoing fish oils, the omega-3 fatty acid oil comprises ≥90 wt % of the omega-3 fatty acids EPA, DHA, docosapentaenoic acid (“DPA”), stearidonic acid (“SDA”), heneicosapentaenoic acid (“HPA”), eicosatetraenoic acid (“ETA”), and alpha-linolenic acid (“ALA”); and preferably 80 to 88 wt % EPA and DHA, 43 to 49.5% EPA and 34.7 to 40.3 wt % DHA.

In some embodiments, the omega-3 fatty acid oil as described in the USP monograph for omega-3 acid ethyl esters (USP33-NF28 2nd supplement, released Jun. 30, 2009, official date Feb. 1, 2011; The United States Pharmacopeial Convention; Rockville, MD.).

The anti-inflammatory plant extract within the terms of this invention are extracts of liquid or dry condition of plants or their parts which are obtained by using water or other solvent. The extracts are preferably in the dry state (“dry extract”), which can preferably be obtained from the fresh or dried parts above ground of euphrasia officinalis such as leaves, blossoms and/or stalks. The term “liquid extract” shall be understood within the terms of the invention as extracts of plants or their parts which are produced by using solvents such as water and/or alcohols such as ethanol, expressed juices from plants or solutions of dried plants in a solvent mixture such as an alcohol-water mixture.

The term “Euphrasia officinalis” (“eyebright”) is understood within the terms of this invention as all species of the genus Euphrasia, e.g. Euphrasia stricta, Euphrasia montana and/or Euphrasia rostkoviana. Other suitable extracts include the extracts of common sage (Salvia officinalis), cowslip (Primula veris), Althaea officinalis, neem oil, turmeric extract, willowbark extract, Cipadessa baccifera extract, black seed oil, and anise extract, among others.

In preferred embodiments, the carotenoid is trans-lutein and/or zeaxanthin isomers, most preferably both.

Preferred embodiments contain beta-carotene.

Non-limiting examples of suitable palatability enhancer include yeast, tallow, rendered animal meals (e.g., poultry, beef, lamb, and pork), flavor extracts or blends (e.g., grilled beef), animal digests, and the like.

The composition of the present invention is preferably substantially free of ascorbic acid.

A further embodiment provides a palatable composition for promoting the eye health of a companion animal comprising: fish oil; safflower oil; Euphrasia officinalis extract; Althaea officinalis powder; and Tagetes erecta extract. It preferably also contains beta-carotene; and a salmon palatability enhancer.

Preferably the palatable composition comprises: between 4 and 16% by weight of fish oil; between 2 and 8% by weight of safflower oil; between 0.5 and 2% by weight of Euphrasia officinalis extract; between 0.25 and 1.2% by weight of Althaea officinalis powder; between 1 and 4% by weight of Tagetes erecta extract; between 0.01 and 0.05% by weight of beta-carotene; and between 2.5 and 10% by weight of a salmon palatability enhancer.

The water content of a preferred embodiment is less than 6% by weight and has a water activity of less than or equal to 0.7 at 25° C., most preferably between 3 and 6% by weight water.

Also provided is a method for promoting eye health in a feline or canine companion animal comprising administering to said animal a composition comprising: an amount of fish oil sufficient to provide at least 50 mg eicosapentaenoic acid (EPA) and at least 25 mg docosapentaenoic acid (DHA); at least 100 mg of a plant oil containing linoleic acid; at least 20 mg of one or more anti-inflammatory plant extracts; an amount of Tagetes erecta extract sufficient to provide at least 3 mg trans-lutein and at least 0.5 mg zeaxanthin isomers; at least 100 mcg RAE beta-carotene; and at least 100 mg of a palatability enhancer; wherein, said composition has a water activity of less than or equal to 0.7 at 25° C.

A preferred edible chew typically has a water activity of 0.65 to 0.75, but some embodiments of the edible pet chews disclosed herein have a slightly lower water activity, for example 0.6 to 0.7.

In an embodiment, the chew comprises a body having a substantially cylindrical shape and comprises radial ridges extending from the body parallel to each other along at least a portion of the length of the body, for example an entire length of the body. However, the edible pet chew can have any, and the edible pet chew is not limited to a specific shape.

In another aspect of the present disclosure, a pet treat is provided comprising the edible chew and one or more fillings therein. For example, the pet treat can be a co-extruded product comprising the edible chew and a soft filling surrounded at least on radial sides by the edible chew (e.g. the filling can be exposed at the ends of the chew). The filling may contain additional teeth cleaning compounds, such as Tetrasodium Pyrophosphate (TSPP) and/or breath-freshening compounds.

Non-limiting examples of suitable additional vitamins include vitamin A, any of the B vitamins, vitamin C, vitamin D, vitamin E, and vitamin K, including various salts, esters, or other derivatives of the foregoing. Non-limiting examples of suitable minerals include calcium, phosphorous, potassium, sodium, iron, chloride, boron, copper, zinc, magnesium, manganese, iodine, selenium, and the like. In a particularly preferred embodiment, calcium hydrogen phosphate can be used as a calcium source. Non-limiting examples of suitable preservatives include potassium sorbate, sorbic acid, sodium methyl para-hydroxybenzoate, calcium propionate, propionic acid, and combinations thereof. The antioxidant can provide an aesthetic effect and influence the odor of the edible pet chew, particularly during extended shelf-life. Non-limiting examples of suitable antioxidants include butylated hydroxyanisole (BHA), butylated hydroxytoluene (BHT), vitamin E (tocopherols), and combinations thereof. Non-limiting examples of suitable colorants include FD&C colors, such as blue no. 1, blue no. 2, green no. 3, red no. 3, red no. 40, yellow no. 5, yellow no. 6, and the like; natural colors, such as roasted malt flour, caramel coloring, annatto, chlorophyllin, cochineal, betanin, turmeric, saffron, paprika, lycopene, elderberry juice, pandan, butterfly pea and the like; titanium dioxide; and any suitable food colorant known to the skilled artisan. Non-limiting examples of suitable palatants flavorants include yeast, tallow, rendered animal meals (e.g., poultry, beef, lamb, and pork), flavor extracts or blends (e.g., grilled beef), animal digests, and the like.

While certain embodiments of the present invention have been described and/or exemplified above, various other embodiments will be apparent to those skilled in the art from the foregoing disclosure. The invention is, therefore, not limited to the particular embodiments described and/or exemplified, but is capable of considerable variation and modification without departure from the scope and spirit of the appended claims.

### EXAMPLES

The following examples describe the invention in further detail. These examples are provided for illustrative purposes only, and should in no way be considered as limiting the invention.

### Example 1:4397-080721

| Ingredient | mg/chew | % w/w |
|---|---|---|
| Ascorbic acid | 181.820 | 4.546 |
| Zinc oxide 80% Zn | 3.940 | 0.099 |
| Marigold flower extract | 28.750 | 0.719 |
| (Tagetes erecta) starch beadlet | | |
| Lutein | | |
| Zeaxanthin isomers | | |
| dl-alpha-tocopheryl acetate | 51.320 | 1.283 |
| Sweet potato powder | 600.000 | 15.000 |
| Vegetarian duck type natural | 200.000 | 5.000 |
| flavor | | |
| Tapioca flour | 738.000 | 18.450 |
| Rice flour | 636.170 | 15.904 |
| Shade palatability enhancer | 200.000 | 5.000 |
| Flaxseed meal | 160.000 | 4.000 |
| Sorbic acid | 20.000 | 0.500 |
| Canola oil | 320.000 | 8.000 |
| Glycerin | 280.000 | 7.000 |
| Mixed tocopherols & rosemary | 20.000 | 0.500 |
| Sunflower lecithin | 160.000 | 4.000 |
| Water | 400.000 | 10.000 |

### Example 2:4397-271021

| Ingredient | mg/chew | % w/w |
|---|---|---|
| Ascorbic acid | 227.273 | 5.682 |
| Zinc oxide 80% Zn | 4.150 | 0.104 |
| Marigold flower extract | 75.000 | 1.875 |
| (Tagetes erecta) starch beadlet | | |
| Lutein | | |
| Zeaxanthin isomers | | |
| dl-alpha-tocopheryl acetate | 54.875 | 1.372 |
| Sweet potato powder | 440.000 | 11.000 |
| Vegetarian duck type natural | 200.000 | 5.000 |
| flavor | | |
| Tapioca flour | 998.702 | 24.968 |
| Rice flour | 480.000 | 12.000 |
| Shade palatability enhancer | 200.000 | 5.000 |
| Flaxseed meal | 160.000 | 4.000 |
| Sorbic acid | 20.000 | 0.500 |
| Canola oil | 320.000 | 8.000 |
| Glycerin | 440.000 | 11.000 |
| Mixed tocopherols & rosemary | 20.000 | 0.500 |
| Sunflower lecithin | 160.000 | 4.000 |
| Water | 200.000 | 5.000 |

### Example 3:4398-291121

| Ingredient | mg/chew | % w/w |
|---|---|---|
| Ascorbic acid | 75.758 | 1.894 |
| Eyebright herb extract | 38.824 | 0.971 |
| (Euphrasia Officinalis) | | |
| Marshmallow root powder | 25.300 | 0.633 |
| (Althaea Officinalis L.) | | |
| Fish oil (Omega-3) | 327.273 | 8.182 |
| Safflower oil (linoleic acid, | 171.429 | 4.286 |
| Omega-6) | | |
| Beta-carotene 30% | 1.000 | 0.025 |
| suspension in olive oil | | |
| Carrot powder | 260.000 | 6.500 |
| Salmon palatability enhancer | 200.000 | 5.000 |
| Tapioca flour | 980.416 | 24.510 |
| Rice bran | 840.000 | 21.000 |
| Flaxseed meal | 240.000 | 6.000 |
| Sorbic acid | 20.000 | 0.500 |
| Mixed tocopherols & | 20.000 | 0.500 |
| rosemary | | |
| Sunflower lecithin | 240.000 | 6.000 |
| Glycerin | 400.000 | 10.000 |
| Water | 160.000 | 4.000 |

### Example 4:4398-150322

| Ingredient | mg/chew | % w/w |
|---|---|---|
| Eyebright herb extract | 38.824 | 0.971 |
| (Euphrasia Officinalis) | | |
| Marshmallow root powder | 25.300 | 0.633 |
| (Althaea Officinalis L.) | | |
| Fish oil (Omega-3) | 327.273 | 8.182 |
| Safflower oil (linoleic acid, | 171.429 | 4.286 |
| Omega-6) | | |
| Beta-carotene 30% | 1.000 | 0.025 |
| suspension in olive oil | | |
| Marigold flower extract | 75.000 | 1.875 |
| (Tagetes erecta) starch | | |
| beadlet | | |
| Lutein | | |
| Zeaxanthin isomers | | |
| Carrot powder | 260.000 | 6.500 |
| Salmon palatability enhancer | 200.000 | 5.000 |
| Tapioca flour | 981.174 | 24.529 |
| Rice bran | 840.000 | 21.000 |
| Flaxseed meal | 240.000 | 6.000 |
| Sorbic acid | 20.000 | 0.500 |
| Mixed tocopherols & | 20.000 | 0.500 |
| rosemary | | |
| Sunflower lecithin | 240.000 | 6.000 |
| Glycerin | 400.000 | 10.000 |
| Water | 160.000 | 4.000 |

### Example 5:4398-F22

| Ingredient | mg/chew | % w/w |
|---|---|---|
| Eyebright herb extract | 38.824 | 0.971 |
| (Euphrasia Officinalis) | | |
| Marshmallow root powder | 25.300 | 0.633 |
| (Althaea Officinalis L.) | | |
| Fish oil (Omega-3) | 327.273 | 81.818 |
| Eicosapentaenoic acid | | |
| (EPA) | | |
| Docosahexaenoic acid | | |
| (DHA) | | |
| Safflower oil (linoleic acid, | 171.429 | 4.286 |
| Omega-6) | | |
| Beta-carotene 30% | 1.000 | 0.025 |
| suspension in olive oil | | |
| Marigold flower extract | 75.000 | 1.875 |
| (Tagetes erecta) starch | | |
| beadlet | | |
| Lutein | | |
| Zeaxanthin isomers | | |
| Carrot powder | 260.000 | 6.500 |
| Salmon palatability enhancer | 200.000 | 5.000 |
| Tapioca flour | 901.174 | 22.529 |
| Rice bran | 840.000 | 21.000 |
| Flaxseed meal | 240.000 | 6.000 |
| Sorbic acid | 20.000 | 0.500 |
| Mixed tocopherols & | 20.000 | 0.500 |
| rosemary | | |
| Sunflower lecithin | 240.000 | 6.000 |
| Glycerin | 400.000 | 10.000 |
| Water | 240.000 | 6.000 |

### Example 6

| Ingredient | mg/chew |
|---|---|
| Eyebright herb extract | 30 |
| (Euphrasia Officinalis) | |
| Marshmallow root powder | 23 |
| (Althaea Officinalis L.) | |
| Fish oil (Omega-3) | 150 |
| Eicosapentaenoic acid | 75 |
| (EPA) | |
| Docosahexaenoic acid | 50 |
| (DHA) | |
| Safflower oil (linoleic acid, | 150 |
| Omega-6) | |
| Beta-carotene 30% | 100 mcg |
| suspension in olive oil | RAE |
| Marigold flower extract | 75 |
| (Tagetes erecta) starch | |
| beadlet | |
| Lutein | 5 |
| Zeaxanthin isomers | 1 |
| Carrot powder | 260.000 |
| Salmon palatability enhancer | 200.000 |
| Tapioca flour | 901.174 |
| Rice bran | 840.000 |
| Flaxseed meal | 240.000 |
| Sorbic acid | 20.000 |
| Mixed tocopherols & | 20.000 |
| rosemary | |
| Sunflower lecithin | 240.000 |
| Glycerin | 400.000 |
| Water | 240.000 |

### Example 7: Stability Testing

### Example 7.1: Formula of Example 1, Stored at 25° C./60% RH (60 ct in HDPE Tub)

| | | Vitamin C | Vitamin E | Lutein | Zeaxanthin | Zinc | Aw |
|---|---|---|---|---|---|---|---|
| LC | | 150 | 20 | 5 | 1 | 3 | |
| Units | | mg | mg | mg | mg | mg | |
| Raw | Lower Spec | 150 | 20.0 | 5.00 | 1.00 | 3.00 | |
| Data | Upper Spec | 240 | 33.0 | 10.00 | 2.00 | 4.05 | |
| | 0 months | 162 | 21.7 | 5.85 | 1.33 | 3.13 | 0.721 |
| | 1 months | 192 | 24.2 | 6.59 | 1.51 | | 0.728 |
| | 2 months | 166 | 25.1 | 6.12 | 1.41 | | 0.725 |
| | 3 months | 159 | 23.5 | 6.28 | 1.46 | | 0.724 |
| | 4 months | 139 | 24.4 | 6.31 | 1.46 | | 0.710 |
| | 5 months | 130 | 24.6 | 6.18 | 1.45 | | 0.722 |
| | 6 months | 121 | 23.5 | 5.68 | 1.28 | 3.01 | 0.723 |
| | 9 months | | 22.8 | 5.50 | 1.34 | | 0.722 |
| | 12 months | | 21.7 | 5.56 | 1.34 | 3.34 | 0.724 |
| | 18 months | | | | | | |
| | 24 months | | | | | | |

### Example 7.2: Formula of Example 1, Stored at 25° C./60% RH (60 ct in Paperboard Tube)

| | | Vitamin C | Vitamin E | Lutein | Zeaxanthin | Zinc | Aw |
|---|---|---|---|---|---|---|---|
| LC | | 150 | 20 | 5 | 1 | 3 | |
| Units | | mg | mg | mg | mg | mg | |
| Raw | Lower Spec | 150 | 20.0 | 5.00 | 1.00 | 3.00 | |
| Data | Upper Spec | 240 | 33.0 | 10.00 | 2.00 | 4.05 | |
| | 0 months | 178 | 22.6 | 6.42 | 1.48 | 3.53 | 0.709 |
| | 1 months | 168 | 24.2 | 6.11 | 1.42 | | 0.704 |
| | 2 months | 119 | 23.9 | 5.52 | 1.31 | | 0.694 |
| | 3 months | 103 | 22.1 | 5.44 | 1.29 | | 0.688 |
| | 4 months | 65 | 23.6 | 5.10 | 1.22 | | 0.668 |
| | 5 months | 63 | 23.3 | 4.77 | 1.13 | | 0.652 |
| | 6 months | 45 | 23.6 | 4.97 | 1.13 | 3.43 | 0.636 |
| | 9 months | | 23.9 | 4.19 | 1.05 | | 0.654 |
| | 12 months | | 22.3 | 4.19 | 1.04 | 3.57 | 0.627 |
| | 18 months | | | | | | |
| | 24 months | | | | | | |

### Example 7.3: Formula of Example 2, Stored at 25° C./60% RH (60 ct in HDPE Tub)

| | | Vitamin C | Vitamin E | Lutein | Zeaxanthin | Zinc | Aw |
|---|---|---|---|---|---|---|---|
| LC per chew | | 150 | 20 | 5 | 1 | 3 | |
| Units for stab samples | | mg/g | mg/g | mg/g | mg/g | mg/g | |
| Raw | Lower Spec | 37.5 | 5.00 | 1.25 | 0.250 | 0.750 | |
| Data | Upper Spec | 75.0 | 8.25 | 3.13 | 0.625 | 1.013 | |
| | 0 months | 56.7 | 5.13 | 2.68 | 0.542 | 0.832 | 0.600 |
| | 1 months | 54.7 | 5.10 | 2.65 | 0.561 | | 0.588 |
| | 1 months | | | 2.30 | 0.508 | | |
| | 2 months | 52.5 | 5.38 | 2.64 | 0.570 | | 0.596 |
| | 3 months | 50.2 | 5.52 | 2.67 | 0.529 | | 0.573 |
| | 4 months | 48.9 | 5.62 | 2.63 | 0.560 | | 0.583 |
| | 5 months | 46.7 | 5.36 | 2.58 | 0.525 | | 0.607 |
| | 6 months | 45.9 | 5.51 | 2.59 | 0.547 | | 0.584 |
| | 9 months | 41.3 | 5.22 | 2.51 | 0.570 | | 0.609 |
| | 12 months | | 5.40 | 2.53 | 0.557 | 0.863 | 0.588 |
| | 18 months | | | | | | |
| | 24 months | | | | | | |

### Example 7.4: Formula of Example 1, Stored at 4° C./75% RH (60 ct in HDPE Tub)

| | | Vitamin C | Vitamin E | Lutein | Zeaxanthin | Zinc | Aw |
|---|---|---|---|---|---|---|---|
| LC | | 150 | 20 | 5 | 1 | 3 | |
| Units | | mg | mg | mg | mg | mg | |
| Raw | Lower Spec | 150 | 20.0 | 5.00 | 1.00 | 3.00 | |
| Data | Upper Spec | 240 | 33.0 | 10.00 | 2.00 | 4.05 | |
| | 0 months | 162 | 21.7 | 5.85 | 1.33 | 3.13 | 0.721 |
| | 1 months | 131 | 23.0 | 5.87 | 1.38 | | 0.720 |
| | 2 months | 40 | 22.9 | 5.14 | 1.33 | | 0.723 |
| | 3 months | 67 | 23.8 | 5.46 | 1.40 | | 0.729 |
| | 4 months | 34 | 23.8 | 5.32 | 1.37 | | 0.756 |
| | 5 months | 20 | 25.6 | 5.39 | 1.37 | | 0.734 |
| | 6 months | 2.07 | 20.7 | 4.27 | 1.11 | 2.87 | 0.734 |

### Example 7.5: Formula of Example 1, Stored at 49° C./75% RH (60 ct in Paperboard Tube)

| | | Vitamin C | Vitamin E | Lutein | Zeaxanthin | Zinc | Aw |
|---|---|---|---|---|---|---|---|
| LC | | 150 | 20 | 5 | 1 | 3 | |
| Units | | mg | mg | mg | mg | mg | |
| Raw | Lower Spec | 150 | 20.0 | 5.00 | 1.00 | 3.00 | |
| Data | Upper Spec | 240 | 33.0 | 10.00 | 2.00 | 4.05 | |
| | 0 months | 178 | 22.6 | 6.42 | 1.48 | 3.53 | 0.709 |
| | 1 months | 83 | 21.3 | 5.44 | 1.32 | | 0.734 |
| | 2 months | 88 | 23.5 | 5.87 | 1.45 | | 0.750 |
| | 3 months | 16 | 20.3 | 4.59 | 1.29 | | 0.755 |
| | 4 months | 6 | 21.1 | 4.44 | 1.25 | | 0.767 |
| | 5 months | 0 | 19.4 | 4.19 | 1.16 | | 0.758 |
| | 6 months | 0 | 20.4 | 3.85 | 1.11 | 3.51 | 0.757 |

### Example 7.6: Formula of Example 2 40° C./75% RH (60 ct in HDPE Tub)

| | | Vitamin C | Vitamin E | Lutein | Zeaxanthin | Zinc | Aw |
|---|---|---|---|---|---|---|---|
| LC per chew | | 150 | 20 | 5 | 1 | 3 | |
| Units for stab samples | | mg/g | mg/g | mg/g | mg/g | mg/g | |
| Raw | Lower Spec | 37.5 | 5.00 | 1.25 | 0.250 | 0.750 | |
| Data | Upper Spec | 75.0 | 8.25 | 3.13 | 0.625 | 1.013 | |
| | 0 months | 56.7 | 5.13 | 2.68 | 0.542 | 0.832 | 0.600 |
| | 1 months | 45.8 | 5.20 | 2.37 | 0.546 | | 0.587 |
| | 2 months | 34.4 | 5.39 | 2.16 | 0.495 | | 0.613 |
| | 3 months | 27.9 | 5.25 | 2.24 | 0.474 | | 0.608 |
| | 4 months | 15.8 | 5.00 | 2.06 | 0.470 | | 0.597 |
| | 5 months | 16.5 | 5.09 | 2.19 | 0.490 | | 0.607 |
| | 6 months | 11.7 | 5.19 | 2.12 | 0.529 | | 0.620 |

### Example 7.7: Formula of Example 3, Stored at 25° C./60% RH (60 ct in HDPE Tub)

| | | Vitamin A | Vitamin C | Omega-3 | EPA-FFA | DHA-FFA | Omega-6 | Aw |
|---|---|---|---|---|---|---|---|---|
| LC per chew | | 200 | 50 | 150 | 75 | 50 | 150 | |
| Units for stab samples | | mcg/g | mg/g | mg/g | mg/g | mg/g | mg/g | |
| Raw | Lower Spec | 50.0 | 12.5 | 37.5 | 18.8 | 12.5 | 37.5 | |
| Data | Upper Spec | 100.0 | 25.0 | 75.0 | 37.5 | 25.0 | 93.8 | |
| | 0 months | 69.1 | 17.9 | 59.0 | 22.4 | 15.1 | 72.9 | 0.565 |
| | 1 months | 69.6 | 14.5 | 58.8 | 21.8 | 14.9 | 78.3 | 0.567 |
| | 2 months | 68.8 | 12.8 | 62.0 | 22.8 | 16.1 | 82.3 | 0.552 |
| | 3 months | 68.9 | 12.5 | 60.8 | 22.2 | 15.4 | 80.1 | 0.555 |
| | 4 months | 70.5 | 11.4 | 60.6 | 22.4 | 15.5 | 82.2 | 0.560 |
| | 5 months | 71.4 | 10.6 | 59.7 | 22.0 | 15.2 | 79.3 | 0.565 |
| | 6 months | 75.1 | 9.8 | 65.8 | 24.3 | 16.7 | 87.6 | 0.567 |
| | 9 months | 71.4 | 7.3 | 65.9 | 24.4 | 16.5 | 90.2 | 0.574 |
| | 12 months | | | | | | | |
| | 18 months | | | | | | | |
| | 24 months | | | | | | | |

### Example 7.8: Formula of Example 3, Stored at 40° C./75% RH (60 ct in HDPE Tub)

| | | Vitamin A | Vitamin C | Omega-3 | EPA-FFA | DHA-FFA | Omega-6 | Aw |
|---|---|---|---|---|---|---|---|---|
| LC per chew | | 200 | 50 | 150 | 75 | 50 | 150 | |
| Units for stab samples | | mcg/g | mg/g | mg/g | mg/g | mg/g | mg/g | |
| Raw | Lower Spec | 50.0 | 12.5 | 37.5 | 18.8 | 12.5 | 37.5 | |
| Data | Upper Spec | 100.0 | 25.0 | 75.0 | 37.5 | 25.0 | 93.8 | |
| | 0 months | 69.1 | 17.9 | 59.0 | 22.4 | 15.1 | 72.9 | 0.565 |
| | 1 months | 69.5 | 10.3 | 58.1 | 21.6 | 14.8 | 77.7 | 0.578 |
| | 2 months | 67.5 | 8.3 | 60.8 | 22.4 | 15.8 | 78.8 | 0.567 |
| | 3 months | 73.8 | 5.1 | 59.6 | 21.8 | 15.5 | 79.0 | 0.569 |
| | 4 months | 78.0 | 3.6 | 60.6 | 22.4 | 15.3 | 79.9 | 0.572 |
| | 5 months | 75.4 | 2.2 | 59.8 | 22.1 | 15.1 | 79.5 | 0.583 |
| | 6 months | 69.5 | | 69.0 | 25.4 | 17.2 | 93.6 | 0.583 |
| | 9 months | | | | | | | |
| | 12 months | | | | | | | |
| | 18 months | | | | | | | |
| | 24 months | | | | | | | |

### Example 7.9: Formula of Example 4, Stored at 25° C./60% RH (60 ct in HDPE Tub)

| | | Vitamin A | Omega-3 | EPA-FFA | DHA-FFA | Omega-6 | Lutein | Zeaxanthin | Aw |
|---|---|---|---|---|---|---|---|---|---|
| LC per chew | | 200 | 150 | 75 | 50 | 150 | 5 | 1 | |
| Units for stab | | mcg/g | mg/g | mg/g | mg/g | mg/g | mg/g | mg/g | |
| samples | | | | | | | | | |
| Raw | Lower Spec | 50.0 | 37.5 | 18.8 | 12.5 | 37.5 | 1.25 | 0.250 | |
| Data | Upper Spec | 100.0 | 75.0 | 37.5 | 25.0 | 93.8 | 3.13 | 0.625 | |
| | 0 months | 76.6 | 60.4 | 23.0 | 16.8 | 76.4 | 2.71 | 0.570 | 0.538 |
| | 1 months | 74.4 | 59.2 | 22.3 | 16.2 | 76.1 | 2.77 | 0.582 | 0.539 |
| | 2 months | 75.8 | 66.9 | 24.9 | 18.3 | 85.5 | 2.72 | 0.554 | 0.543 |
| | 3 months | 68.5 | 64.3 | 24.4 | 17.4 | 80.5 | 2.78 | 0.569 | 0.555 |
| | 4 months | 90.1 | 65.5 | 24.5 | 17.6 | 85.8 | 2.66 | 0.572 | 0.551 |
| | 5 months | 69.8 | 66.3 | 24.8 | 18.1 | 86.7 | 2.61 | 0.560 | 0.550 |
| | 6 months | 72.3 | 60.8 | 22.7 | 16.6 | 79.2 | | | 0.556 |
| | 9 months | 75.2 | 58.5 | 21.8 | 16.0 | 76.3 | | | 0.544 |
| | 12 months | | | | | | | | |
| | 18 months | | | | | | | | |
| | 24 months | | | | | | | | |

### Example 8

Twenty male and female beagles identified by cage number and ear tattoo were presented with test compositions on an individual bases. A single piece of the composition of Example 1 was offered in the stainless steel bowls once daily for 2 days. Bowls were presented for 5 minutes. Two hours before the test compositions were offered, all dogs were presented with 300 grams of Purina Dog Chow for 30 minutes. Total test composition consumption was measured and recorded.

### Example 9

Twenty male and female beagles identified by cage number and ear tattoo were presented with test compositions on an individual bases. A single piece of the composition of Example 2 was offered in the stainless steel bowls once daily for 2 days. Bowls were presented for 5 minutes. Two hours before the test compositions were offered, all dogs were presented with 300 grams of Purina Dog Chow for 30 minutes. Total test composition consumption was measured and recorded.

### Example 10

Twenty male and female beagles identified by cage number and ear tattoo were presented with test compositions on an individual bases. A single piece of the composition of Example 3 was offered in the stainless steel bowls once daily for 2 days. Bowls were presented for 5 minutes. Two hours before the test compositions were offered, all dogs were presented with 300 grams of Purina Dog Chow for 30 minutes. Total test composition consumption was measured and recorded.

### Example 11

Twenty male and female beagles identified by cage number and ear tattoo were presented with test compositions on an individual bases. A single piece of the composition of Example 4 was offered in the stainless steel bowls once daily for 2 days. Bowls were presented for 5 minutes. Two hours before the test compositions were offered, all dogs were presented with 300 grams of Purina Dog Chow for 30 minutes. Total test composition consumption was measured and recorded.

### Example 12

Twenty male and female beagles identified by cage number and ear tattoo were presented with test compositions on an individual bases. A single piece of the composition of Example 5 was offered in the stainless steel bowls once daily for 2 days. Bowls were presented for 5 minutes. Two hours before the test compositions were offered, all dogs were presented with 300 grams of Purina Dog Chow for 30 minutes. Total test composition consumption was measured and recorded.

1. A palatable composition for promoting the eye health of a companion animal comprising:
a. omega-3 fatty acid oil;
b. at least one anti-inflammatory plant extract; and
c. at least one carotenoid.

2. The palatable composition claimed in claim 1, wherein said omega-3 fatty acid oil contains an omega-3 fatty acid selected from the group consisting of eicosapentaenoic acid (EPA) and docosapentaenoic acid (DHA).

3. The palatable composition claimed in claim 2, wherein said omega-3 fatty acid oil comprises at least two omega-3 fatty acids; wherein a first omega-3 fatty acid is eicosapentaenoic acid (EPA) and a second omega-3 fatty acid is docosapentaenoic acid (DHA).

4. The palatable composition claimed in claim 2, wherein said omega-3 fatty acid oil further comprises an omega-6 fatty acid.

5. The palatable composition claimed in claim 4, wherein said omega-6 fatty acid is linoleic acid.

6. The palatable composition claimed in claim 1, wherein said anti-inflammatory plant extract is selected from the group consisting of Euphrasia officinalis extract and Althaea officinalis powder.

7. The palatable composition claimed in claim 5, comprising at least two anti-inflammatory plant extract; wherein a first anti-inflammatory plant extract is Euphrasia officinalis extract and a second anti-inflammatory plant extract is Althaea officinalis powder.

8. The palatable composition claimed in claim 1, wherein said carotenoid is selected from the group consisting of trans-lutein and zeaxanthin isomers.

9. The palatable composition claimed in claim 7, comprising at least two carotenoids; wherein a first carotenoid is trans-lutein and a second carotenoid is zeaxanthin isomers.

10. The palatable composition claimed in claim 8, further comprising beta-carotene.

11. The palatable composition claimed in claim 1, further comprising a palatability enhancer.

12. The palatable composition claimed in claim 11, wherein said palatability enhancer is an animal derived composition.

13. The palatable composition claimed in claim 1, wherein said composition is substantially free of ascorbic acid.

14. A palatable composition for promoting the eye health of a companion animal comprising:
a. fish oil;
b. safflower oil;
c. Euphrasia officinalis extract;
d. Althaea officinalis powder; and
e. Tagetes erecta extract.

15. The palatable composition claimed in claim 14, further comprising
f. beta-carotene; and
g. a salmon palatability enhancer.

16. The palatable composition claimed in claim 15, wherein said composition is substantially free of ascorbic acid.

17. The palatable composition claimed in claim 15, comprising:
a. between 4 and 16% by weight of fish oil;
b. between 2 and 8% by weight of safflower oil;
c. between 0.5 and 2% by weight of Euphrasia officinalis extract;
d. between 0.25 and 1.2% by weight of Althaea officinalis powder;
e. between 1 and 4% by weight of Tagetes erecta extract;
f. between 0.01 and 0.05% by weight of beta-carotene; and
g. between 2.5 and 10% by weight of a salmon palatability enhancer.

18. The palatable composition claimed in claim 17, wherein the water content is less than 6% by weight and has a water activity of less than or equal to 1.0000 at 25° C.

19. The palatable composition claimed in claim 18, wherein the composition has a water activity of less than or equal to 0.7 at 25° C.

20. The palatable composition claimed in claim 19, further comprising sorbic acid.

21. The palatable composition claimed in claim 17, wherein said composition is substantially free of ascorbic acid.

22. The palatable composition claimed in claim 17, further comprising carrot powder in an amount of at least 3% by weight.

23. A method for promoting eye health in a feline or canine companion animal comprising administering to said animal a composition comprising:
a. an amount of fish oil sufficient to provide at least 50 mg eicosapentaenoic acid (EPA) and at least 25 mg docosapentaenoic acid (DHA);
b. at least 100 mg of a plant oil containing linoleic acid;
c. at least 20 mg of one or more anti-inflammatory plant extracts;
d. an amount of Tagetes erecta extract sufficient to provide at least 3 mg trans-lutein and at least 0.5 mg zeaxanthin isomers;
e. at least 100 mcg RAE beta-carotene; and
f. at least 100 mg of a palatability enhancer;
wherein, said composition has a water activity of less than or equal to 1.0 at 25° C.

24. The method claimed in claim 22, wherein the composition further comprises at least 10 mg sorbitol.
